(19) United States
(12) Patent Application Publication
(10) Pub. No.: US 2008/0096856 A1
(43) Pub. Date: Apr. 24, 2008

(54) Antibiotic Compound

(75) Inventors: John F. Barrett, Branchburg, NJ (US); Kithsiri B. Herath, Kendall Park, NJ (US); Hiranthi Jayasuriya, Edison, NJ (US); Prakash S. Masurekar, Warren, NJ (US); John G. Ondeyka, Fanwood, NJ (US); Sheo Bux Singh, Edison, NJ (US); Chaowei Zhang, Paramus, NJ (US)

Correspondence Address: MERCK AND CO., INC, P O BOX 2000, RAHWAY, NJ, 07065-0907, US

(73) Assignee: MERCK & CO., LTD., Rahway, NJ (US)

(21) Appl. No.: 11/661,466

(22) PCT Filed: Sep. 16, 2005
(86) PCT No.: PCT/US05/33326

§ 371 Date: Feb. 26, 2007

Related U.S. Application Data
(60) Provisional application No. 60/611,951, filed on Sep. 22, 2004.

(51) Int. Cl. A61K 31/33; A61P 43/00; C07D 513/22; C12P 17/18

(52) U.S. Cl. 514/183; 435/119; 540/456

(57) ABSTRACT

Fermentation of a nutrient medium with a Nocardia spp. yields a novel broad spectrum antibiotic compound of structural formula (I) or a pharmaceutically acceptable salt, ester, enantiomer, diasteriomer or mixture.

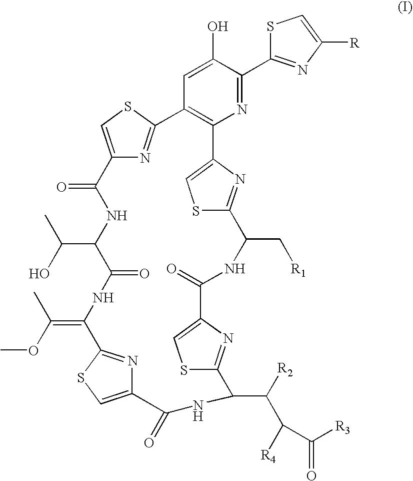

### CROSS REFERENCE

This case claims the benefit of provisional application U.S. Ser. No. 60/611,951, filed on Sep. 22, 2004.

### BACKGROUND OF THE INVENTION

The present invention relates to broad spectrum thiazolyl-peptide antibiotic compounds that are useful in treating bacterial infections.

Infections caused by bacteria are a growing medical concern as many of these bacteria are resistant to various antibiotics. Such microbes include Staphylococcus aureus, Staphylococcus hemolyticus, Pediococcus spp., and Streptococcus pyogenes, Streptococcus pneumoniae, Pseudomonas aeruginosa, Vibrio cholerae, Vibrio parahemolyticus, Actinobacter calcoaeticus, Stenotrophomonas maltophilia. The antibiotic of this invention, thus comprises an important contribution to therapy for treating infections which are resistant to various known antibiotics.

A prior art compound that is also a thiazolyl-peptide and which is used for treating bacterial infections is thiostrepton, GE2270A, nocathiacins, glycothiohexide, and nosiheptide.

In the present invention, the thiazolyl-peptide antibiotics are produced from a Nocardia spp. fermentation and possesses antibacterial activity against bacterial infections that are sensitive and resistance to currently available antibiotics.

### SUMMARY OF THE INVENTION

This invention is concerned with novel thiazolyl-peptide antibiotics of the formula I: 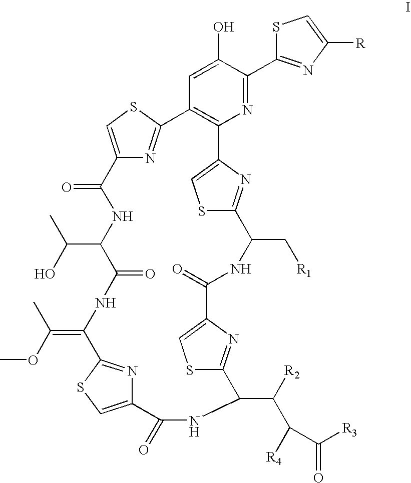
or a pharmaceutically acceptable salt, ester, enantiomer, diasteriomer or mixture thereof,
wherein:
R represents —C(O)NH₂; —C(O)NHC═CH₂C(O)NH₂;
R₁, R₂, and R₃ independently represent OH;
or R₁, R₂ and R₃ taken together represent: 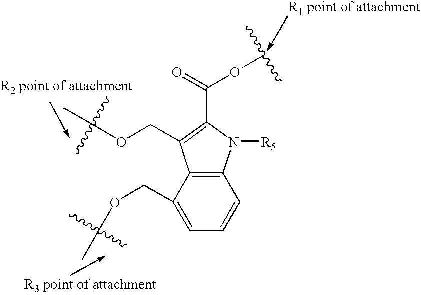
R₄ represents OH, 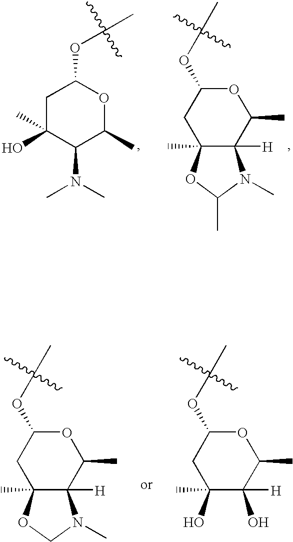
and R₅ represents hydrogen or OH.

The invention is also concerned with a process for the production of the compound of formula I by fermentation with a Nocardia spp. The invention is also concerned with a process for isolating the compound of formula I from the fermentation broth.

### DETAILED DESCRIPTION OF THE INVENTION

This invention is also concerned with the compounds of formula I in particular compounds of structural formula Ia, Ib, Ic, Id, Ie, If, Ig, Ih, and Ii: 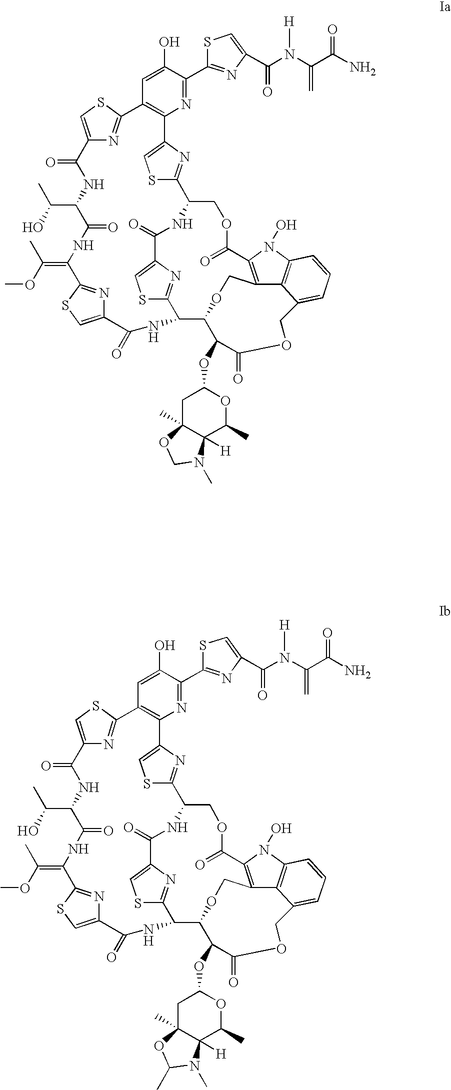 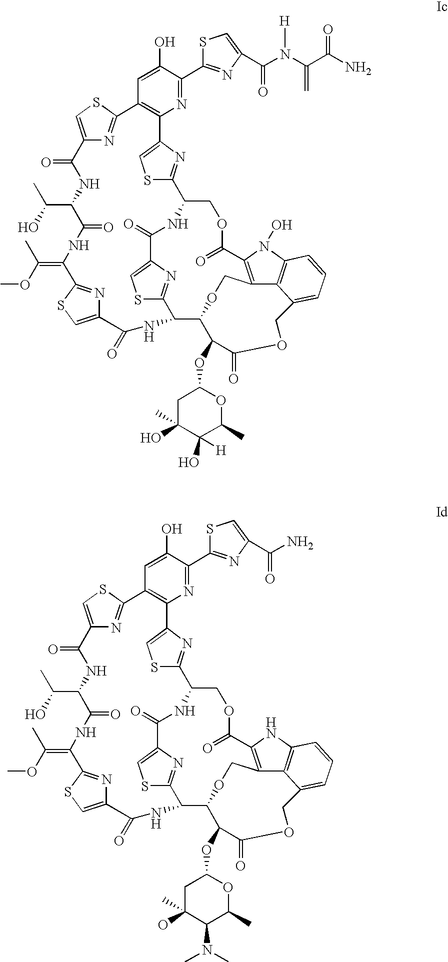 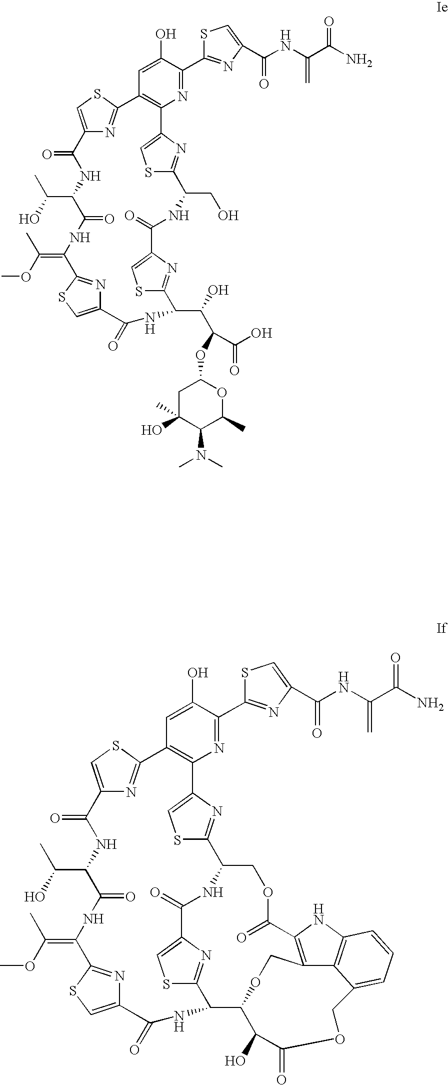 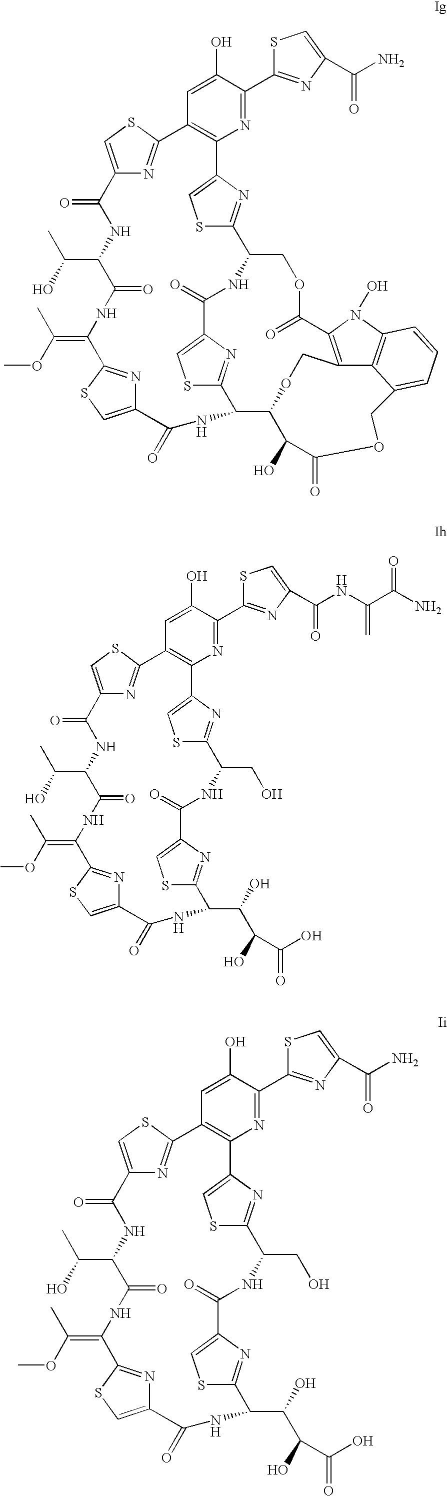
or pharmaceutically acceptable salts, esters, enantiomers, diastereomers or a mixture thereof.

The pharmaceutically acceptable salts of the compounds of this invention include the conventional non-toxic salts as formed, from non-toxic inorganic or organic bases. For example, such conventional non-toxic salts include those derived from inorganic bases such as an alkali or alkaline earth metal hydroxide, e.g., potassium, sodium, lithium, calcium, or magnesium, and the like: and the salts prepared from organic bases such as an amine, e.g., dibenzylethylene-diamine, trimethylamine, piperidine, pyrrolidine, benzylamine and the like, or a quaternary ammonium hydroxide such as tetramethylammonium hydroxide and the like.

The pharmaceutically acceptable salts can be synthesized from the compounds of this invention by conventional chemical methods. Generally, the salts are prepared by reacting the free acid with stoichiometric amounts or with an excess of the desired salt-forming inorganic or organic base in a suitable solvent or various combinations of solvents.

The compounds of this invention are a broad spectrum antibiotic useful in the treatment of bacterial infections. They demonstrate antibacterial activity primarily against S. aureus, E. faecalis, E. faecium, S. pneumoniae, B. subtilus including species that are resistant to many known antibiotics. The minimum inhibitory concentration (MIC) values for these test strains range from 0.01 to less than 200 ug/mL for test strains such as Staphylococcus aureus, Staphylococcus hemolyticus, Streptococcus pyogenes, Streptococcus pneumoniae, and E. faecalis. The compounds of the invention can be formulated in pharmaceutical compositions by combining the compounds with a pharmaceutically acceptable carrier. Examples of such carriers are set forth below.

The compounds may be employed in powder or crystalline form, in liquid solution, or in suspension. They may be administered by a variety of means; those of principal interest include: topically, orally and parenterally by injection (intravenously or intramuscularly).

Compositions for injection, one route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the Compound, the virulence of the infection and other factors. Such matters, however, are left to the routine discretion of the physician according to principles of treatment well known in the antibacterial arts.

The compositions for administration to humans per unit dosage, whether liquid or solid, may contain from about 0.01% to as high as about 99% of Compound I, one embodiment of the range being from about 10-60%. The composition will generally contain from about 2 mg to about 2.5 g of Compound I, one embodiment of this range being from about 2 mg to 1000 mg. In parenteral administration, the unit dosage will typically include pure Compound I in sterile water solution or in the form of a soluble powder intended for solution, which can be adjusted to neutral pH and isotonicity.

The invention described herein also includes a method of treating a bacterial infection in a mammal in need of such treatment comprising the administration of the compound of formula I to the mammal in an amount effective to treat the infection.

One embodiment of the methods of administration of a compound of formula I includes oral and parenteral methods, e.g., i.v. infusion, i.v. bolus and i.m. injection.

For adults, about 0.1-50 mg of a compound of formula I per kg of body weight given one to four times daily is preferred. The preferred dosage is 2 mg to 1000 mg of the antibacterial given one to four times per day. More specifically, for mild infections a dose of about 5-200 mg two or three times daily is recommended. For moderate infections against highly susceptible gram positive organisms a dose of about 20-1000 mg three or four times daily is recommended. For severe, life-threatening infections against organisms at the upper limits of sensitivity to the antibiotic, a dose of about 100-2000 mg three to four times daily may be recommended.

For children, a dose of about 0.1-50 mg of a compound of formula I per kg of body weight given one to four times daily is typically recommended. Another aspect of this invention is realized when the dosage is 2 mg to 1000 mg of the antibacterial given one to four times per day.

Another aspect of this invention is the process for producing the compounds of formula I which comprises cultivating a Nocardia sp. microorganism in a suitable nutrient medium and then recovering the compound of this invention from the fermentation broth. The organism in question is was obtained from the American Type Culture Collection (ATCC), 12301 Parklawn Drive, Rockville, Md., 20852 registered with accession number ATCC 202099. It is deposited in Merck culture collection with an accession number of MA7332. Any restrictions relating to public access to the microorganism shall be irrevocably removed upon patent issuance. Although the use of this particular species is described in connection with this invention, there may be other species and mutants of the above organism capable of producing Compound I, and their use is contemplated in carrying out the process of this invention.

The compounds of structural formula I are produced by the aerobic fermentation of a suitable medium under controlled conditions via inoculation with a culture of the ATCC accession number 202099. The suitable medium is preferably aqueous and contains sources of assimilable carbon, nitrogen, and inorganic salts.

The medium employed for fermentation primarily the well-known Difco Tryptic Soy Broth, either alone or with added nutrients is commonly used by those skilled in the art.

It should be noted that the nutrient media described herein are merely illustrative of the wide variety of media which may be employed and are not intended to limit the scope of this invention in any way.

The fermentation is conducted at temperatures ranging from about 10° C. to about 40° C.; however for optimum results it is preferred to conduct the fermentation at about 31-32° C. The pH of the nutrient medium during the fermentation can be about 5.5 to about 7.5.

It is to be understood that for the fermentative production of the compound of this invention, the invention is not limited to the use of the particular Nocardia spp. with ATCC accession number 202099. It is especially desired and intended that there be included in the scope of this invention the use of other natural or artificial mutants produced or derived from the described cultures, or other variants or species of the Nocardia genus insofar as they can produce the compound of this invention. The artificial production of mutant species or strains of Nocardia from ATCC 202099 may be achieved by conventional, physical or chemical mutagens, for example, ultraviolet irradiation of the described culture, or nitrosoguanidine treatment and the like. Recombinant DNA techniques such as protoplast fusion, plasmid incorporation, chromosome fragment incorporation and the like also may prove useful.

### EXAMPLE 1

Step 1a: Fermentation For Production Of Compounds

Ia, Ic, Id, Ie, If, Ih, and Ii. A 1 mL frozen vegetative stock culture of Nocardia sp. ATCC 202099 (MA7332) was used to inoculate 50 mL of seed medium, in a 250 mL flask, containing the following components per liter of water: starch, 20 g; dextrose, 5 g; N-Z amine (Kerry Bio-Science, Hoffman Estates, Ill.), 3 g; yeast extract, 2 g; Pharmamedia (Traders Protein, Memphis, Tenn.), 5 g; calcium carbonate, 1 g. The culture was incubated at 32° C. on a rotary shaker operating at 220 rpm for 3 days. Twenty mL of the resulting culture was used to inoculate 500 mL of seed medium, in a 2 L flask, containing the same components as for the 50 mL culture listed above. The culture was incubated at 32° C. on a rotary shaker operating at 180 rpm for 1 day. The resulting 500 mL culture was used to inoculate 20 L of media, in a 30 L fermenter, containing the following components per liter of water: dextrose, 20 g; peptone, 5 g; primary yeast, 10 g; Allophosite (aluminum silicate), 5 g; P2000 anti-foam (is a polymeric material that prevents foaming made by Dow Chemical, Midland, Mich.), 1 mL. The production fermentation was operated at a temperature of 32° C., a back-pressure of 5 psi, and an agitation rate of 300 rpm. Air was sparged through the fermenter at 10 slpm and pH was controlled at 7.0 with NaOH and H₂SO₄. The fermenter was operated for 13 days at which time the culture was harvested compounds were extracted and isolated as described in Step 2a.

Step 1b-1: Fermentation for Production of Compound Ib:

A 1 mL frozen vegetative stock culture of Nocardia sp. ATCC 202099 (MA7332) was used to inoculate 50 mL of seed medium, in a 250 mL flask, containing the following components per liter of water: starch, 20 g; dextrose, 5 g; N-Z amine, 3 g; yeast extract, 2 g; Pharmamedia, 5 g; calcium carbonate, 1 g. The culture was incubated at 32° C. on a rotary shaker operating at 220 rpm for 2 days. Twenty mL of the resulting culture was used to inoculate 500 mL of seed medium, in a 2 L flask, containing the same components as for the 50 mL culture listed above. The culture was incubated at 32° C. on a rotary shaker operating at 180 rpm for 2 days. The resulting 500 mL culture was used to inoculate 15 L of media, in a 23 L fermenter, containing the following components per liter of water: soluble starch, 25 g; glucose, 15 g; acid hydrolyzed casein 7.5 g; yeast extract, 12 g; soybean meal, 3.5 g; beef extract, 3.5 g; anti-foam (P2000), 1 mL. The production fermentation was operated at a temperature of 32° C., a back-pressure of 5 psi, and an agitation rate of 300 rpm. Air was sparged through the fermenter at 5 standard liters per minute (slpm) and pH was controlled at 7.0 with NaOH and H₂SO₄. The fermenter was operated for 10 days at which time the culture was harvested and extracted as described below for isolation of compound Ib.

Step 1b-2: Fermentation for Isolation of Compound Ig:

A 1 mL frozen vegetative stock culture of Nocardia sp. ATCC 202099 (MA7332) was used to inoculate 50 mL of seed medium, in a 250 mL flask, containing the following components per liter of water: starch, 20 g; dextrose, 5 g; N-Z amine, 3 g; yeast extract, 2 g; Pharmamedia, 5 g; calcium carbonate, 1 g. The culture was incubated at 32° C. on a rotary shaker operating at 220 rpm for 2 days. Twenty mL of the resulting culture was used to inoculate 500 mL of seed medium, in a 2 L flask, containing the same components as for the 50 mL culture listed above. The culture was incubated at 32° C. on a rotary shaker operating at 180 rpm for 2 days. The resulting 500 mL culture was used to inoculate 20 L of media, in a 30 L fermenter, containing the following components per liter of water: dextrose, 20 g; peptone, 5 g; primary yeast, 10 g; Allophosite (aluminum silicate), 5 g; P2000 anti-foam (is a polymeric material that prevents foaming), 2 mL. The production fermentation was operated at a temperature of 32° C., a back-pressure of 5 psi, and an agitation rate of 300 rpm. Air was sparged through the fermenter at 10 slpm and pH was controlled at 7.0 with NaOH and H₂SO₄. The fermenter was operated for 10 days at which time the culture was harvested and extracted with ethyl acetate for recovery of compound Ig.

Step 2a: Isolation Of Compounds Ia, Ic, Id, Ie, If, Ib, and Ii.

A 18 liter fermentation broth (pH=5.0) was extracted twice with 18 and 16 liters each of ethyl acetate by shaking overnight at room temperature. The ethyl acetate layers were separated, pooled and concentrated under reduced pressure to give 7.1 grams of solid. The aqueous layer was filtered through celite and the cake was extracted twice with 8 liters of acetone each. Pooled acetone extracts were concentrated to dryness to give 15.6 grams of solid which was placed on a sintered funnel and washed with hexane (4×150 mL) providing 12.8 grams of solid which was suspended in 250 mL of 1:1 methylene chloride-methanol and filtered. An aliquot of 35 grams of Sephadex LH20 was poured on to the filtrate and solvent was removed under reduced pressure. The LH20 powder coated with the compounds was charged on top of a 2.0 liter Sephadex LH20 column packed in 1:4 hexane-methylene chloride. The column was eluted with two column volume each of 1:4 hexane-methylene chloride (fraction 3), 2.5% methanol-methylene chloride (fractions 4-6), 1.5 column volume of 5% methanol-methylene chloride (fractions 7-11), one column volume of 10% methanol-methylene chloride (fractions 12-15), 1.5 column volumes each of 20 (fraction 16-18), 50 (19-21) and finally with 100% methanol. Fractions were pooled based on analysis on TLC and analytical HPLC affording fractions 1-21 as listed in parentheses with the elution solvents.

ISOLATION OF COMPOUND Ia: LH20 fraction 3 (1 gram) was dissolved in 50% methanol-methylene chloride and adsorbed on to 5 g of silica gel 60 (230-400 mesh, E-M Scientific, Germany). It was dried under vacuum and purified using vacuum liquid chromatography on 100 g of silica gel 60 (230-400 mesh, E-M Scientific, Germany) in a 20 cm ID sintered funnel. This was eluted with 0.5 L of chloroform, followed by one L of 10% methanol-chloroform, 0.5 L of 20% methanol-chloroform, one L of 50% methanol-chloroform and finally washed with 5% ammonium hydroxide in 50% methanol-chloroform. Fractions from the 10% methanol-chloroform elution was concentrated under reduced pressure and lyophilized to yield 650 mg of a solid material which was purified by preparative HPLC in 13 identical runs using Zorbax SB Phenyl [(21×250 mm) eluting with a 36 min gradient of 40 to 50% acetonitrile-water containing 0.1% TFA, at a flow rate of 12 mL/min. Compound Ia eluted at 32-33 minutes. It was lyophilized to yield 42 mg of light yellow powdery material which was further purified by two preparative HPLC's using a shallower gradient on Zorbax SB Phenyl (21×250 mm eluting with a 42 min gradient of 40 to 50% acetonitrile-water containing 0.1% TFA, at 12 mL/min]. Compound Ia eluted in 33-37 minutes. The fractions were pooled and lyophilized to yield compound Ia as a pale yellow powder.

ISOLATION OF COMPOUND Ic: LH-20 fraction 07 that eluted in 5% methanol in methylene chloride was concentrated to dryness to give 400 mg of material that was subjected to a silica gel chromatography in a pre-packed sintered glass funnel (40 g silica gel, EM science, particle size 230-240 mm, eluted with acetic acid/methanol/chloroform, 1:0:99, 1:1:98, 1:2:97, 1:3:96, 1:4:95, 100 mL for each, 1:5:94 (5×100 mL), then ammonium hydroxide/methanol/chloroform, 1:7.5:91.5 (100 mL), 1:10:89 (3×100 mL), 1:25:74 (2×100 mL), 1:99:0 (100 mL). Fractions eluting with the last 300 mL of 1:5:94 acetic acid/methanol/chloroform elution solvent were pooled and adjusted to pH=7.0 with ammonium hydroxide, concentrated to dryness, triturated with water, filtered, and the filtrate was concentrated to dryness to afford 33.5 mg of material, which was chromatographed on a preparative-HPLC (YMC ODS-AQ, 20×250 mm, 5 mL/min, 40-50% aqueous acetonitrile with 0.05% TFA over 60 min, 215 nm). The fraction eluting at 46 minutes was lyophilized to furnish compound Ic.

ISOLATION OF COMPOUND Id: A 15 L fermentation of Nocardia sp grown in 23 L fermentation tank for 7 days as described earlier was extracted with 15 L ethyl acetate and filtered. The filtrate was concentrated under reduced pressure using rotary evaporator and lyophilized to produce 15 g of oily material. Dried ethyl acetate extract (15 g) was dissolved in 50 mL of 90% methanol-methylene chloride and charged on to a 2 L Sephadex L1420 column and was eluted with methanol-methylene chloride (3:1) at a flow rate of 5 mL/min. Forty mL fractions were collected. Compounds of interest eluted in fraction 21-36 (0.8-1.4 column volumes). These fractions were pooled and concentrated to dryness to give 4.5 g of oily material. This was dissolved in 50% methanol-methylene chloride and adsorbed on to 12 g of silica gel 60 (230-400 mesh, E-M Scientific, Germany). It was dried under vacuum and purified using vacuum liquid chromatography on 100 g of silica gel 60 (230-400 mesh, E-M Scientific, Germany) in a 8.5 cm ID sintered funnel. This was washed with 2 L of methanol-water-methylene chloride (3:0.025:97) and compound Id eluted with 2 L of methanol-water-methylene chloride (35:0.025:65). It was concentrated under reduced pressure and lyophilized to yield 1.8 g of solid material which was dissolved in 10% methanol-methylene chloride and adsorbed on to 3 g of Diol (DL12S50, 120A, s-50 μm YMC Co. Ltd. Japan). It was dried under vacuum and purified on a 150 g of Diol (DL12S50, 120A, s-50 μm YMC Co. Ltd. Japan) column (1.75×8 inch) at the flow rate of 5 mL/min. This was eluted with methylene chloride, followed by methanol-water-methylene chloride (1:0.5:99) solvent system with increasing percentages of methanol ending with methanol-water-methylene chloride (15:1:84) and finally washed with (10:1:89) methanol-ammonium hydroxide-dichloromethane, 50 mL fractions were collected. The fractions eluting with last solvent were pooled to provide 48 mg of semi-purified fraction which was dissolved in 10% methanol-methylene chloride and pre-adsorbed on to 0.5 g of silica gel 60 (230-400 mesh, E-M Scientific, Germany) and purified on 10 g of silica gel 60 (230-400 mesh, E-M Scientific, Germany) column (0.5×8.5 inch) using flow rate of 1.5 mL/min. This was eluted with 250 mL of chloroform, followed by chloroform-ammonium hydroxide with increasing percentage of methanol [i.e. methanol-ammonium hydroxide-chloroform 2.5:0.125:97.5 (600 mL), 3:0.125:97 (800 mL), 5:0.125:95 (800 mL), 10:0.125:90 (1.4 L), 15:0.125:85 (600 mL), 25:0.125:75 (200 mL), 40: 0.125:60 (200 mL)]. Fraction containing compound Id eluted with 10:0.125:90 (methanol-ammonium hydroxide-chloroform). They were pooled and concentrated under reduced pressure and lyophilized to afford 4 mg of compound Id.

ISOLATION OF COMPOUND Ie: LH20 fraction 16 (432 mg) was dissolved in chloroform/methanol (1:1) and pre adsorbed onto 2 g silica gel and solvents were removed under reduced pressure and the powder was applied on to a silica gel-packed sintered glass funnel (25 g, EM science, particle size 230-240 mm, house vacuum, eluted by methanol/chloroform, 2.5%, 5.0%, 7.5%, 10%, 15%, 20%, 30%, 50%, 75%, 100 mL each, finally, 100% methanol wash 400 mL. The methanol fraction was concentrated to give 84.4 mg of material which was subjected to prep-HPLC fractionation (YMC ODS-AQ, 20×250 mm, 10 mL/min, 25-35% acetonitrile without TFA over 40 min, 215 nm). HPLC fraction eluting at 25 minutes was lyophilized to furnish compound Ie as powder.

ISOLATION OF COMPOUND If: LH-20 fraction 07 that eluted in 5% Methanol in Methylene chloride was concentrated to dryness to give 400 mg of material that was subjected to a silica gel chromatography in a pre-packed sintered glass funnel (40 g silica gel, EM science, particle size 230-240 mm, eluted with acetic acid/Methanol/Chloroform, 1:0:99, 1:1:98, 1:2:97, 1:3:96, 1:4:95, 100 mL for each, 1:5:94 (5×100 mL), then ammonium hydroxide/Methanol/Chloroform, 1:7.5:91.5 (100 mL), 1:10:89 (3×100 mL), 1:25:74 (2×100 mL), 1:99:0 (100 mL). Fractions eluting with last 300 mL of 1:5:94 acetic acid/Methanol/Chloroform elution solvent were pooled and pH was adjusted to 7.0 with ammonium hydroxide, concentrated to dryness, triturated with water, filtered, and the filtrate was concentrated to dryness to afford 33.5 mg of material, which was chromatographed on a preparative-HPLC (YMC ODS-AQ, 20×250 mm, 5 mL/min, 40-50% aqueous acetonitrile with 0.05% TFA over 60 min, 215 nm). The fraction eluting at 31 min was lyophilized to furnish compound If.

ISOLATION OF COMPOUND Ih and Ii: LH-20 fraction 21 (175.0 mg) eluted with 50% methanol-methylene chloride was subjected to preparative-HPLC using YMC ODS-AQ, 20×250 mm column at a flow rate of 10 mL/min with a 40 minutes 25-35% aqueous acetonitrile gradient containing 0.05% TFA detecting at 214 nm. Fractions eluting at 22 and 31 minutes were lyophilized to afford compounds II and Ih, respectively, as powders.

ISOLATION OF COMPOUND Ib: The 100 L broth from 6 tanks as described in step 1b-1 was extracted with 50 L ethyl acetate. The top ethyl acetate layer was removed, and the aqueous layer was filtered with celite. The ethyl acetate extract was concentrated to dryness and triturated with hexane to obtain 10 g of material. The filtered cake was extracted with 2×20 L acetone. After removal of acetone, precipitated compounds were filtered through a sintered funnel. Solids from ethyl acetate and acetone extracts were pooled and washed with hexane (500 mL) and dissolved in 1:1 methanol/methylene chloride (500 mL) and filtered through a sintered funnel. The filtrate was concentrated to dryness to give 42 grams of solid that was dissolved in 1:1 methylene chloride/methanol (200 mL), and pre adsorbed onto 50 g silica gel and applied to a 12×17 cm sintered glass funnel packed with 550 g silica gel and eluted sequentially with 1% methanol/chloroform with 1% acetic acid 2 L, 2% methanol/chloroform with 1% acetic acid 1 L, 3% methanol/chloroform with 1% acetic acid 1 L, 4% methanol/chloroform with 1% acetic acid 1 L, 5% methanol/chloroform with 1% acetic acid 4 L, 6% methanol/chloroform with 1% acetic acid 1 L, 7.5% methanol/chloroform with 1% acetic acid 3 L, 7.5% methanol/chloroform with 1% ammonium hydroxide 5 L, 15% methanol/chloroform with 1% ammonium hydroxide 2 L, 30% methanol/chloroform with 1% ammonium hydroxide 2 L, 60% methanol/chloroform with 1% ammonium hydroxide 2 L, 100% methanol with 1% ammonium hydroxide 3 L (500 mL/fraction, total 54 fractions).

Fraction 30 was concentrated to dryness to give 146.5 mg of a powder which was subjected to prep-HPLC (Zorbax SB-phenyl, 21.2×250 mm, 12 mL/min, 40-50% acetonitrile in water with 0.1% TFA over 45 min, 214 nm). Fractions eluting at 39-40 min were pooled and lyophilized to give compound Ib. ISOLATION OF COMPOUND Ig: Fourteen liters of broth of step 1 b-2 was extracted with 14 liters of ethyl acetate. The mixture was filtered and the cells extracted twice with 2 liters each of acetone and combined. The acetone extract was concentrated under vacuum to 1.5 liters of aqueous leading to precipitation which was filtered to give 5.8 grams of solid. A five gram aliquot of the solid was dissolved in methanol-methylene chloride and 5 grams of silica gel was added. The solvent was removed under reduced pressure to give a powder which was added on top of a 250 gram silica bed in a 2-liter sintered funnel in 99-1 chloroform-acetic acid. The elution was carried out with 3 volumes each of 99-1 chloroform-acetic acid, 99-1-1; chloroform-methanol-acetic acid followed by 98-2-1, 97-3-1, 96-4-1, 95-5-1, 90-10-1 and 90-10-1 chloroform-methanol-ammonium hydroxide. Fractions 17 and 18 eluting with solvent compositions 95-5-1 were combined (105 mg), dissolved and added to 3-5 g of silica gel and concentrated to dryness. This was added to the top of a 15 g silica gel dry packed column and eluted with 30 column volumes of 97-3-1; chloroform-methanol-water for a total of 155, 6 mL each fractions. Fractions were analyzed first by silica TLC and selected fraction assayed by HPLC. Fractions 21-50 (20 mg) were added to 1 g of silica gel and concentrated to dryness. This was added on to the top of a 15 g silica gel dry packed column and eluted with 48 column volumes of 98-2-1; chloroform-methanol-water for a total of 240, 6 mL each fractions. Fractions 180 to 210 were combined and concentrated to dryness to give 3.2 mg of mostly Ig which was further purified on Zorbax phenyl semi-prep HPLC column (9.4×250 mm) with multiple injections using a 21 minute 40-60% aqueous acetonitrile gradient with a 2 min hold. The fractions eluting at 14 minutes were combined and lyophilized to afford compound Ig.

Step 3: Physiochemical Properties of Ia-Ie

The structure of Compound Ia, Ib, Ic, Id, Ie, If, Ig, Ib and Ii was determined by the use of mass spectroscopy, ¹H NMR and ¹³C NMR.

Compound Ia:

Molecular weight: 1434

Molecular formula: C₆₁H₅₈N₁₄O₁₈S₅

HRESIMS (m/z): 1435.2696 (observed for M+H), 1435.2734 (Calculated)

UV (methanol) λₘₐₓ (log ε) 222 (4.77), 288 (4.40), 358 (4.08) nm

IR (neat) νₘₐₓ 3389, 3096, 2929, 1669, 1638, 1535, 1476, 1424, 1387, 1320, 1253, 1203, 1229, 1099, 1038, 1016 cm⁻¹

¹H NMR (CDCl3+CD3OD) δ ppm δ 8.38 (1H, d, J=10 Hz), 8.31 (1H, s), 8.27 (1H, s), 8.21 (1H, s), 8.08 (1H, d, J=11.5 Hz), 7.93 (1H, s), 7.76 (1H, d, J=8.5 Hz), 7.68 (1H, s), 7.64 (1H, s), 7.36 (1H, t, J=8.5 Hz), 7.16 (1H, d, J=7.0 Hz), 6.58 (1H, s), 5.96 (1H, d, J=12.0 Hz), 5.93 (1H, d, J=10.0 Hz), 5.70 (1H, m), 5.59 (1H, s), 5.26 (1H, m), 5.04 (1H, t, J=7.0 Hz), 4.97 (1H, d, J=5.0 Hz), 4.91 (1H, d, J=12.5 Hz), 4.84 (TH, d, J=10.0 Hz), 4.40 (1H, d, J=9.5 Hz), 4.24 (1H, m), 4.19 (1H, d, J=5.5 Hz), 4.16 (1H, d, J=12.0 Hz), 3.87 (1H, d, J=6.0 Hz), 3.81 (3H, s), 3.56 (1H, d, J=10.5 Hz), 2.91 (1H, m), 2.70 (1H, s), 2.37 (1H, dd, J=16.0, 5.5 Hz), 1.90 (3H, s), 1.83 (1H, dd, J=15.0, 7.0 Hz), 1.40 (3H, s), 1.39 (3H, d, J=6.0 Hz), 0.72 (3H, d, J=6.5 Hz).

¹³C NMR (CDCl3+CD3OD) δ 172.0, 169.7, 169.4, 167.8, 166.6, 166.2, 165.5, 162.3, 162.0, 161.7, 161.6, 160.6, 159.5, 158.8, 155.3, 151.7, 149.9, 149.8, 149.3, 146.1, 144.1, 135.5, 134.8, 133.1, 130.3, 127.9, 126.9, 126.6, 125.7, 125.4, 124.9, 124.3, 124.0, 120.5, 119.6, 112.4, 110.5, 110.1, 104.3, 93.9, 84.8, 81.6, 72.0, 70.0, 68.3, 66.3, 64.7, 64.2, 64.2, 56.5, 55.9, 41.3, 35.9, 25.4, 18.2, 16.3, 13.8.

Compound Ib:

Molecular Weight: 1448

Molecular Formula: C₆₂H₆₀N₁₄O₁₈S₅

UV (methanol+THF, 1:1) λₘₐₓ 222 (ε52736), 288 (ε23110), 362 (ε9383)

IR (ZnSe) νₘₐₓ 3387, 2978, 1742, 1667 (br, strong), 1531, 1475, 1422, 1320, 1252, 1200, 1128, 1097, 1070, 1025, 832, 801, 721 cm⁻¹

HRESIMS: 1449.2930 (observed for M+H), 1449.2892 (Calculated for M+H)

¹H NMR (CDCl3+CD3OD) δ (ppm): 8.39 (1H, d, J=10.8 Hz), 8.33 (1H, brs), 8.24 (1H, brs), 8.21 (1H, s), 8.10 (1H, d, J=10.9 Hz), 7.93 (1H, s), 7.94 (1H, brs), 7.76 (1H, d, J=7.2 Hz), 7.69 (1H, s), 7.62 (1H, brs), 7.37 (1H, t, J=7.0 Hz), 7.13 (1H, d, J=7.0 Hz), 6.57 (1H, brs), 5.98 (1H, m), 5.96 (1H, m), 5.67 (1H, dd, J=10.1, 5.1 Hz), 5.58 (1H, brs), 5.27 (1H, dd, J=10.1, 5.1 Hz), 5.07 (1H, t, J=6.3 Hz), 4.93 (11H, d, J=12.3 Hz), 4.84 (1H, d, J=10.1 Hz), 4.46 (1H, brs), 4.43 (1H, d, J=9.1 Hz), 4.41 (1H, d, J=10.9 Hz), 4.22 (1H, brs), 4.17 (1H, d, J=10.3 Hz), 3.93 (1H, q, J=6.5 Hz), 3.81 (1H, d, J=10.0 Hz), 3.80 (3H, s), 3.00 (1H, brs), 2.94 (1H, brs), 2.73 (3H, s), 2.41 (1H, dd, J=15.7, 5.5 Hz), 1.89 (3H, s), 1.80 (1H, dd, J=15.7, 7.8 Hz), 1.45 (3H, brs), 1.40 (3H, brs), 1.39 (3H, s), 0.75 (3H, d, J=6.1 Hz).

¹³C NMR (CDCl3+CD3OD) δ (ppm) 171.4, 169.3, 169.1, 167.4, 166.3, 165.9, 165.1, 162.1, 161.7, 161.4, 161.3, 160.3, 159.2, 158.4, 155.0, 151.3, 149.6, 149.5, 148.9, 145.8, 143.8, 135.2, 134.4, 132.8, 129.9, 127.5, 126.6, 126.3, 126.3, 125.4, 124.9, 124.3, 124.0, 123.7, 120.1, 119.3, 111.9, 110.1, 109.7, 103.7, 93.1, 91.9, 81.0, 79.3, 72.6, 69.2, 67.1, 65.9, 64.1, 64.1, 63.5, 56.0, 55.3, 48.7, 48.4, 39.6, 35.4, 24.8, 17.5, 15.1, 14.5, 13.3.

Compound Ic:

Molecular Weight: 1409

Molecular Formula: C₅₉H₅₅N₁₃O₁₉S₅

HRESIMS: 1410.2429 (observed for M+H), 1410.2419 (Calculated for M+H)

¹H NMR (CDCl3+CD3OD) δ 8.31 (1H, s), 8.29 (1H, s), 8.24 (1H, s), 8.00 (1H, s), 7.83 (1H, d, J=8.6 Hz), 7.69 (1H, s), 7.66 (1H, s), 7.42 (1H, dd, J=8.6, 7.0 Hz), 7.16 (1H, d, J=7.0 Hz), 6.62 (1H, s), 6.07 (1H, d, J=12.3 Hz), 5.81 (1H, brd, J=9.1 Hz), 5.69 (1H, dd, J=10.2, 5.4 Hz), 5.54 (1H, s), 5.30 (1H, dd, J=11.3, 5.9 Hz), 4.95 (1H, d, J=12.3 Hz), 4.90 (1H, d, J=10.7 Hz), 4.89 (1H, d, J=4.3 Hz), 4.44 (1H, dd, J=11.3, 9.7 Hz), 4.42 (1H, d, J=10.2 Hz), 4.28 (1H, d, J=4.3 Hz), 4.23 (1H, d, J=10.7 Hz), 3.91 (1H, d, J=10.2 Hz), 3.83 (3H, s), 3.35 (1H, m), 3.05 (1H, d, J=9.7 Hz), 2.93 (1H, m), 2.17 (1H, d, J=13.9 Hz), 1.91 (3H, s), 1.89 (1H, d, J=13.9 Hz), 1.49 (3H, s), 1.39 (3H, d, J=6.4 Hz), 0.67 (3H, d, J=6.4 Hz).

Compound Id:

Molecular Weight: 1351

Molecular Formula: C₅₈H₅₈N₁₃O₁₆S₅

ESIMS: 1352 (observed for M+H)

¹H NMR (CDCl₃+CD₃OD) δ (ppm) 8.44 (1H, d, J=9.5 Hz), 8.38 (1H, s), 8.29 (1H, s), 8.25 (1H, s), 8.00 (1H, s), 7.83 (1H, d, J=10.5 Hz), 7.74 (1H, s), 7.71 (1H, d, J=8.5 Hz), 7.60 (1H, s), 7.39 (2H, m), 7.16 (1H, d, J=9.0 Hz), 6.15 (1H, d, J=12.5 Hz), 5.82 (1H, brd, J=8.0 Hz), 5.76 (1H, dd, J=11.4 Hz), 5.38 (1H, dd, J=11, 4.5 Hz), 5.16 (1H, d, J=10 Hz), 5.05 (1H, d, J=5 Hz), 4.95 (1H, d, J=12.5 Hz), 4.65 (1H, d, J=11.5 Hz), 4.43 (1H, d, J=9.5 Hz), 4.30 (1H, m), 4.21 (1H, d, J=10 Hz), 3.99 (1H, brd, J=6 Hz), 3.94 (1H, dd, J=9.5, 1.5 Hz), 3.86 (3H, s), 2.60 (6H, s), 2.29 (1H, m), 2.07 (1H, d, J=14.5 Hz), 1.97 (1H, m), 1.95 (3H, s), 1.57 (3H, s), 1.32 (3H, d, J=6 Hz), 0.78 (3H, d, J=7 Hz)

Compound Ie:

Molecular Weight: 1253

Molecular Formula: C₅₀H₅₅N₁₃O₁₆S₅

UV (methanol+THF, 1:1) λₘₐₓ 224 (ε60244), 364 (ε11502)

IR (ZnSe) νₘₐₓ 3368, 1657 (br, strong), 1536, 1479, 1423, 1321, 1251, 1124, 1025, 887, 761 cm⁻¹

HRESIMS: 1254.2551 (observed for M+H), 1254.2571 (Calculated for M+H)

¹H NMR (DMSO-d₆) δ (ppm): 10.08 (1H, s, NH), 9.55 (1H, brs, NH), 8.61 (1H, brs, NH), 8.55 (1H, s), 8.51 (1H, d, J=8.8 Hz, NH), 8.43 (1H, s), 8.35 (1H, s), 8.19 (1H, s), 8.05 (1H, brs, NH), 7.59 (1H, brs, NH), 7.87 (1H, s), 7.81 (1H, s), 7.38 (1H, brs, NH), 5.72 (1H, brs), 6.35 (1H, brs), 5.66 (1H, dd, J=9.3, 2.7 Hz), 5.26 (1H, ddd, J=9.3, 9.3, 4.4 Hz), 5.12 (1H, brs), 4.78 (1H, brs), 4.66 (1H, d, J=7.9 Hz), 4.55 (1H, brs), 4.02 (1H, m), 4.00 (1H, m), 3.88 (3H, s), 3.72 (1H, m), 3.50 (1H, t, J=10.2 Hz), 3.70 (1H, m), 2.60 (6H, s), 2.36 (1H, brs), 2.02 (3H, s), 1.88 (1H, dd, J=13.6, 3.4 Hz), 1.74 (1H, d, J=13.6 Hz), 1.46 (3H, s), 1.30 (3H, d, J=5.7 Hz), 0.95 (3H, d, J=6.2 Hz).

¹³C NMR (DMSO-D6) δ (ppm) 171.7, 169.3, 169.2, 168.8, 166.7, 165.1, 164.2, 162.2, 160.8, 160.5, 160.1, 160.0, 158.5, 153.6, 151.5, 149.9, 149.4, 149.2, 146.6, 141.6, 135.1, 134.3, 129.5, 127.2, 127.1, 125.4, 124.8, 123.0, 118.4, 110.2, 103.5, 94.4, 72.7, 72.4, 69.4, 68.4, 67.2, 65.1, 62.1, 56.0, 55.7, 52.9, 49.6, 44.1, 40.1, 30.1, 20.9, 18.2, 12.7

Compound If:

Molecular Weight: 1249

Molecular Formula: C₅₂H₄₃N₁₃O₁₅S₅

HRESIMS (m/z): 1250.1670 (observed for M+H), 1250.1683 (Calculated for M+H)

¹H NMR (CDCl₃-CD₃OD) δ (ppm) 10.7 (1H, brs, NH), 9.7 (1H, s, NH), 8.32 (1H, s), 8.31 (1H, d, J=9.7 Hz), 8.23 (1H, s), 8.18 (1H, s), 8.09 (1H, brs, OH), 7.92 (s), 7.81 (1H, d, J=10.8 Hz), 7.72 (1H, s), 7.61 (1H, d, J=8.5 Hz), 7.57 (1H, s), 7.30 (1H, m), 7.30 (1H, d, J=7.5 Hz), 7.07 (1H, d, J=7.0 Hz), 5.99 (1H, d, J=12.3 Hz), 4.93 (1H, d, J=12.6 Hz), 5.95 (1H, dd, J=9.6, 1.8 Hz), 5.70 (1H, dd, J=11.0, 3.9 Hz), 5.57 (1H, d, J=1.7 Hz), 6.52 (1H, d, J=1.8 Hz), 5.31 (1H, dd, J=11.8, 4.4 Hz), 4.59 (1H, d, J=11.4 Hz), 5.07 (1H, d, J=10.1 Hz), 4.08 (1H, d, J=10.0 Hz), 4.21 (1H, brd, J=7.4 Hz), 4.20 (1H, d, J=9.3 Hz), 3.74 (3H, s), 3.65 (1H, dd, J=9.6, 1.9 Hz), 1.85 (3H, s), 1.85 (1H, m), 1.25 (3H, d, J=6.3 Hz).

¹³C NMR (CDCl₃-CD₃OD) δ (ppm) 174.4, 169.3, 168.2, 168.2, 166.4, 166.3, 165.7, 164.3, 161.8, 161.7, 161.4, 159.2, 159.2, 158.5, 154.9, 151.6, 150.1, 149.4, 149.1, 146.0, 142.6, 136.4, 134.2, 132.8, 129.5, 127.0, 126.3, 125.9, 125.9, 124.3, 124.1, 124.1, 123.6, 123.4, 122.8, 119.3, 115.8, 115.8, 110.0, 103.7, 81.4, 68.0, 67.3, 66.2, 64.4, 62.0, 55.3, 54.6, 50.7, 49.2, 17.6, 12.9.

Compound Ig:

Molecular Weight: 1196

Molecular Formula: C₄₉H₄₀N₁₂O₁₅S₅

HRESIMS (m/z): 1197.1420 (Observed for M+H), 1197.1412 (Calculated for M+H)

¹H NMR (DMSO-d₆): δ (ppm) 10.83 (1H, s), 8.95 (1H, s), 8.64 (1H, s), 8.56 (1H, s), 8.51 (1H, s), 8.45 (1H, s), 8.38 (1H, d, J=8.5 Hz), 8.24 (1H, s), 7.99 (1H, s), 7.91 (1H, d, J=11 Hz), 7.86 (1H, s), 7.71 (1H, s br), 7.70, (1H, s br), 7.38 (1H, d, J=8.0 Hz), 7.34 (1H, t, J=8.0 Hz), 7.17 (1H, d, J=7.0 Hz), 6.08 (1H, d, J=7.0 Hz), 5.92 (1H, d, J=12.0 Hz), 5.87 (1H, d, J=9.5 Hz), 5.75 (1H, s), 5.74 (1H, m), 5.22 (1H, m), 5.02 (1H, d, J=12.0 Hz), 4.69 (1H, d, J=10.5 Hz), 4.53 (1H, m), 4.90, (1H, d, J=10.5 Hz), 4.20 (1H, m), 4.10 (1H, m), 4.02 (1H, dd, 9.5, 7.5), 3.88 (3H, s), 3.73 (1H, d, J=9.5 Hz), 2.53 (1H, m), 12.9 (3H, s), 1.16 (3H, s br).

¹³C NMR (DMSO-d₆) δ (ppm) 174.3, 167.9, 167.9, 167.7, 167.6, 163.8, 162.7, 161.0, 160.9, 160.2, 160.2, 159.1, 157.8, 157.5, 150.9, 150.6, 149.4, 148.6, 145.9, 143.1, 134.8, 134.4, 130.0, 128.4, 127.4, 126.7, 126.2, 125.4, 125.0, 124.0, 122.9, 120.0, 119.4, 112.3, 111.2, 109.7, 81.3, 67.7, 67.1, 65.1, 64.3, 63.1, 56.0, 55.7, 49.5, 49.3, 17.8, 13.0.

Compound Ih:

Molecular Weight: 1082

Molecular Formula: C₄₁H₃₈O₁₄N₁₂S₅

HRESIMS (m/z): 10830.1277 (Observed for M+H), 1083.1312 (Calculated for M+H)

¹H NMR (DMSO-d₆) δ (ppm) 8.59 (1H, s), 8.45 (1H, s), 8.40 (1H, s), 8.20 (1H, s), 7.92 (1H, s), 7.86 (1H, s), 6.34 (1H, s), 5.88 (1H, dd, J=9.7, 2.5 Hz), 5.74 (1H, s), 5.32 (1H, ddd, J=9.5, 9.5, 5.2 Hz), 4.66 (1H, bd, J=8.8 Hz), 4.05 (1H, bs), 3.89 (3H, s), 3.73 (1H, dd, J=11.1, 4.4 Hz), 3.68 (1H, brs), 3.60 (1H, brs), 3.51 (1H, t, J=10.2 Hz), 2.05 (3H, s), 0.90 (3H, d, J=6.5 Hz)

¹³C NMR (DMSO-d₆) δ (ppm) 173.6, 169.4, 169.4, 169.1, 167.1, 165.1, 164.0, 162.2, 160.5, 160.5, 160.4, 160.4, 158.4, 153.4, 150.8, 149.8, 149.8, 149.2, 146.6, 142.6, 135.1, 134.3, 129.6, 127.3, 127.1, 125.7, 125.5, 123.5, 119.0, 110.0, 103.8, 73.6, 71.5, 66.1, 62.2, 56.3, 55.8, 53.1, 49.7, 16.9, 12.7.

Compound Ii:

Molecular Weight: 1013

Molecular Formula: C₃₈H₃₅O₁₃N₁₁S₅

HRESIMS (m/z): 1014.1073 (Observed for M+H), 1014.1097 (Calculated for M+H)

¹H NMR (DMSO-d₆) δ (ppm) 8.56 (1H, s), 8.45 (1H, s), 8.39 (1H, s), 8.19 (1H, s), 7.94 (1H, s), 7.84 (1H, s), 5.86 (1H, brd, J=9.4 Hz), 5.31 (1H, m), 4.65 (1H, bd, 7.3 Hz), 4.03 (1H, m), 3.88 (3H, s), 3.77 (1H, m), 3.73 (1H, m), 3.59 (1H, m), 3.49 (1H, m), 2.03 (3H, s), 0.88 (3H, d, J=6.5 Hz).

The Protocols Used to Determine the Antibacterial Activity of Compound I are Described Below.

Materials:

Cation-Adjusted Mueller Hinton Broth (MH; BBL)

50% Lysed Horse Blood (LHB; BBL) (stored frozen)

RPMI 1640 (BioWhittaker)

Human Serum (Pel-Freez)

RPMI 1640 (BioWhittaker)

Haemophilus Test Medium (HTM, Remel)

Trypticase Soy Broth (TSB, 5 mL/tube; BBL)

0.9% Sodium Chloride (Saline; Baxter)

Trypticase Soy+5% Sheep Blood Agar Plates (TSA; BBL)

Sabouraud Dextrose Agar Plates (BBL)

Chocolate Agar Plates (BBL)

2× Skim Milk (Remel)

Microbank Beads (Kramer Scientific)

MIC 2000 Microtiter plate inoculator.

2× Trypticase Soy Broth (TSB, BBL)+15% glycerol/50% horse serum.

96-Well Microtiter plates, lids, inoculum trays (Dynex Laboratories)

8-Channel Finn Multichannel pipettor, 0.5-10 μL volume

Methods:

Media Preparation

Cation-Adjusted Mueller Hinton Broth (BBL): Prepared according to manufacturer's instructions (22 gms dissolved in 1000 mL water; autoclaved 22 minutes). Stored refrigerated. Filter-sterilized before use using a Corning 0.45™ cellulose acetate filter.

50% Lysed Horse Blood: Defibrinated horse blood is diluted 1:1 with sterile distilled water; frozen, thawed and re-frozen (at least 7 times), then centrifuged. Stored frozen at −20° C.

Cation-Adjusted Mueller Hinton+2.5% Lysed Horse Blood: Aseptically add 5 mL 50% lysed horse blood to 100 mL Cation-Adjusted Mueller Hinton Broth. Filter-sterilize before use using a Corning 0.45™ cellulose acetate filter.

Cation-Adjusted Mueller Hinton+50% Human Serum: Aseptically add 50 mL Human Serum to 50 mL 2× Cation-Adjusted Mueller Hinton Broth. Filter-sterilize before use using a Corning 0.45™ cellulose acetate filter.

Haemophilus Test Medium (Remel): Received prepared from manufacturer. Filter-sterilized before use using a Corning 0.45™ cellulose acetate filter.

0.9% Sodium Chloride (Saline; Abbott Labs): Received prepared from manufacturer.

2× Skim Milk (Remel): Received prepared from manufacturer.

All agar plates are received prepared from manufacturer.

| CONDITIONS AND | |
|---|---|
| INOCULUM | FOR REPRESENTATIVE STRAINS |
| BACILLUS, | INCUBATION CONDITIONS, 35° C.; |
| STAPHYLOCOCCUS, | MICS READ AT 18-22 HOURS; |
| ENTEROCOCCUS: | CATION-ADJUSTED MUELLER HINTON |
| ESCHERICHI:, | (CAMHB; BBL); INOCULUM = 10⁵ |
| | CFU/ML |
| STREP. | INCUBATION CONDITIONS, 35° C.; |
| PNEUMONIAE: | MICS READ AT 22-24 HOURS; |
| | CATION-ADJUSTED MUELLER HINTON + |
| | 2.5% LYSED HORSE BLOOD (LHB); |
| | INOCULUM = 10⁵ CFU/ML |
| HAEMOPHILUS | INCUBATION CONDITIONS, 35° C.; |
| INFLUENZAE: | MICS READ AT 18-22 HOURS; |
| | HAEMOPHILUS TEST MEDIUM (HTM; |
| | REMEL); INOCULUM = 10⁵ CFU/ML |
| CANDIDA: | INCUBATION CONDITIONS, 35° C.; |
| | MICS READ AT 24 HOURS; RPMI 1640 |
| | MEDIUM (BIOWHITTAKER) |
| | INOCULUM = 10³ CFU/ML |

HIGHEST CONCENTRATION OF ANTIBIOTIC TESTED = 64 μG/ML (WHEN STARTING FROM A 1 MG/ML SOL'N IN 50% DMSO)
FINAL CONCENTRATION OF DMSO PER WELL = 3.2%

Selection and Maintenance of Isolates

The strains used are isolates from either the Merck Culture Collection, the Merck Clinical Culture Collection or from Clinical Trials. The strain of Haemophilus influenzae is a mouse pathogen used for in vivo testing at Merck. The Escherichia coli strain is a cell wall permeable strain. The Candida albicans strain is used as a control. These culture are maintained as frozen stocks at −80° C. in a) Microbank beads; b) 2× Skim Milk; or c) in 2× Trypticase Soy Broth+15% glycerol/50% horse serum (Haemophilus and Streplococcus pneumoniae).

Inoculum Preparation

Selected isolates are sub-cultured onto either Chocolate Agar Plates (Haemophilus influenzae), onto Trypticase Soy+5% Sheep Blood Agar Plates (Streptococcus pneumoniae, Staphylococcus aureus, Escherichia coli, Enterococcus, Bacillus) or onto Sabouraud Dextrose Agar (Candida) and incubated at 35° C. Haemophilus and Streptococcus pneumoniae are incubated in 5% CO₂; all other isolates are incubated in ambient air. Isolates are sub-cultured 2× before assay.

Colonies are selected from plates and used to prepare an inoculum equivalent to a 0.5 McFarland standard in Trypticase Soy Broth. An inoculum with a density equivalent to a 1.0 McFarland standard is prepared for Streptococcus pneumoniae. The inoculum density for all cultures is ˜10⁸ CFU/mL in TSB. This TSB inoculum is diluted 1:10 in sterile saline (4 mL inoculum+36 mL saline; equivalent to ˜10⁷ CFU/mL) and kept on ice until used to inoculate microtiter plates.

Colony counts are performed on randomly-selected isolates to confirm CFU/well (TSB inoculum plated out 10⁻⁵, 10⁻⁶ onto either TSA II+5% SB or onto chocolate agar plates, incubated overnight, 35° C., CO₂)

Plate Filling

All wells of 96-well microtiter plates (Dynex) are filled with 100 TL media. Haemophilus test media plates are prepared to test Haemophilus influenzae; Cation-Adjusted Mueller Hinton+5% Lysed Horse Blood plates are prepared to test Streptococcus pneumoniae; Cation-Adjusted Mueller Hinton Broth plates are prepared to test Enterococcus, Staphylococcus aureus, Escherichia coli and Bacillus subtilis. RPMI 1640 is used to test Candida. The MICs against S. aureus Smith are determined in Cation-adjusted Mueller Hinton and in Cation-Adjusted Mueller Hinton+50% Human Serum, to determine if the compound is inactivated by some component in serum (indicated by an increase in the MIC). Filled plates are wrapped in plastic bags (to minimize evaporation), stored frozen and thawed before use.

Preparation of Compounds

The compounds are prepared on a weight basis. Compounds are prepared to 2-10 mg/mL in 100% DMSO, then diluted to 1 mg/mL in a 1:1 dilution of DMSO/2×CAMHB (final concentration=50% DMSO/50% CAMHB). Compounds are serially diluted 1:1 in 50% DMSO/50% CAMHB in BD Biosciences Deep Well Polypropylene 96 well plates (starting concentration 1-5 mg/mL).

Microbroth Dilution Assay

Using a Finn Automated Multichannel Pipette, (0.5-10 μL volume) 6.4 TLs of antimicrobial working solutions are added to wells of filled microtiter plates (concentration of antimicrobial in first well=512-64 microg/mL; concentration of DMSO=3.2%). Antimicrobials are added in this manner to keep constant the amount of DMSO in each well (to keep compounds solubilized and to account for the possibility of non-specific killing by the DMSO. The last row contains a growth control of 3.2% DMSO.

With each assay, controls are run. They are Penicillin G and chloramphenicol, prepared in the same manner as the compounds. Ertapenem is included as a control for the serum protein binding assay.

Plate Inoculation

All wells of microtiter plates are inoculated with (saline-diluted) culture using the MIC 2000 System, an automated plate inoculating device which delivers an inoculum of 1.5 TL per well. Plates are incubated at 35° C. in ambient air. An uninoculated plate is also incubated as a sterility check. Results are recorded after 22-24-hours' incubation. Plates were read to no growth. The MIC is defined as the lowest antimicrobial level which resulted in no growth after 22-24-hours' incubation.

Compounds Ia-Ii demonstrate antibacterial activity against various strains of S. aureus, E. faecalis, E. faecium, B. subtilus and S. pneumoniae. Compound Ia-Ii also demonstrate antibacterial activity against various species that are resistant to many known antibiotics such as methicillin-resistant S. aureus (MRSA), vancomycin-resistant Enterococcus sp. (VRE), multidrug-resistant E. faecium, macrolide-resistant S. aureus and S. epidermidis, and linezolid-resistant S. aureus and E. faecium. The minimum inhibitory concentration (MIC) values for these test strains range from 0.01 to 200 ug/mL. MICs are obtained in accordance to the NCCLS guidelines.

What is claimed is:

1. A compound of structural formula I: 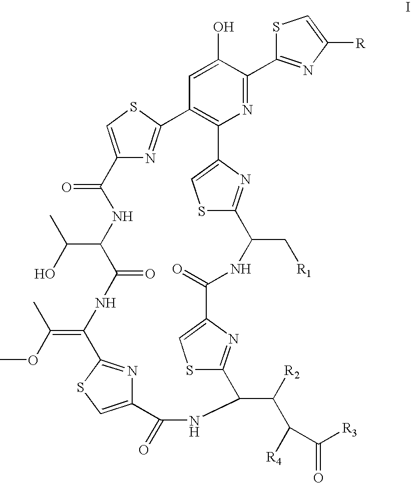
or a pharmaceutically acceptable salt, ester, enantiomer, diasteriomer or mixture thereof,
wherein:
R represents —C(O)NH₂; —C(O)NHC═CH2C(O)NH₂;
R₁, R₂, and R₃ independently represent OH;
or R₁, R₂ and R₃ taken together represent: 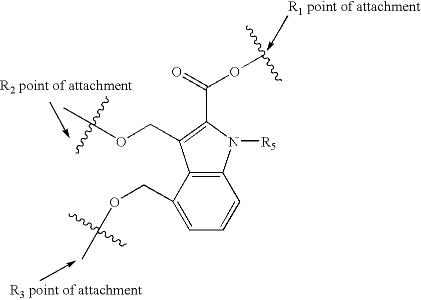
R₄ represents OH, 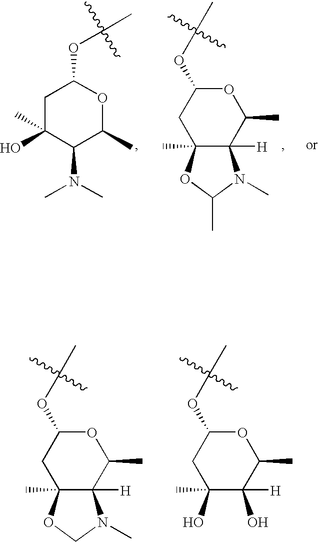
and R₅ represents hydrogen or OH.

2. A compound which is: 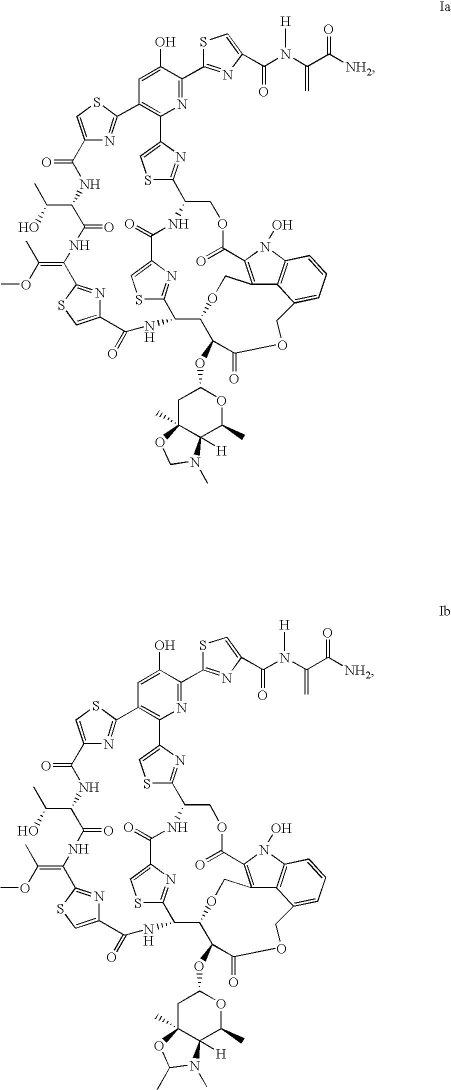 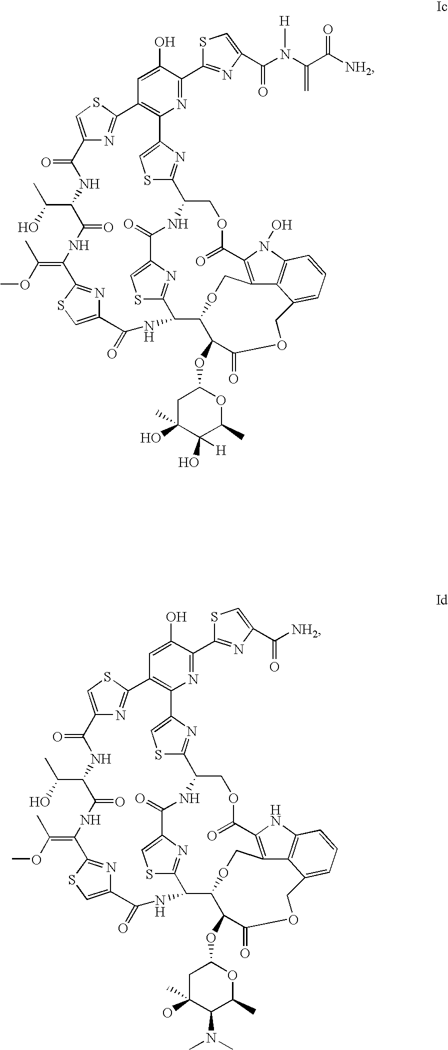 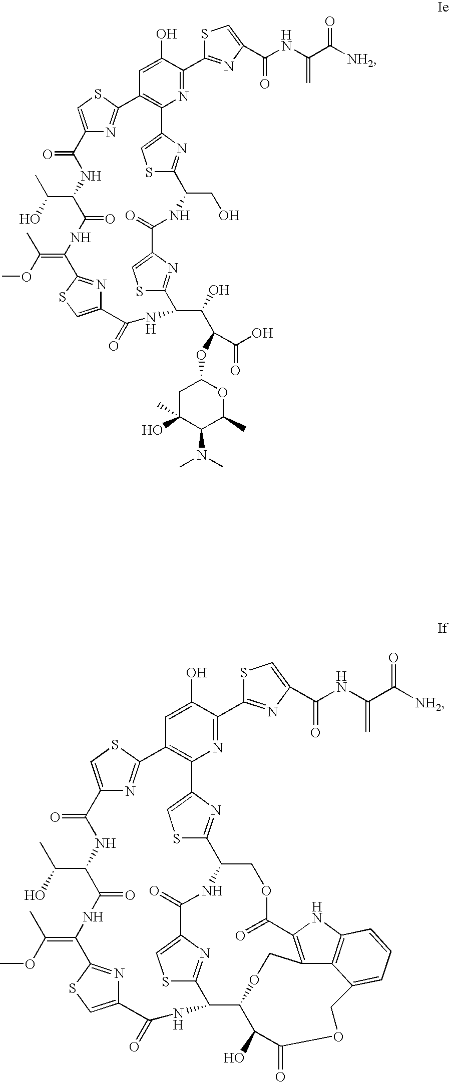 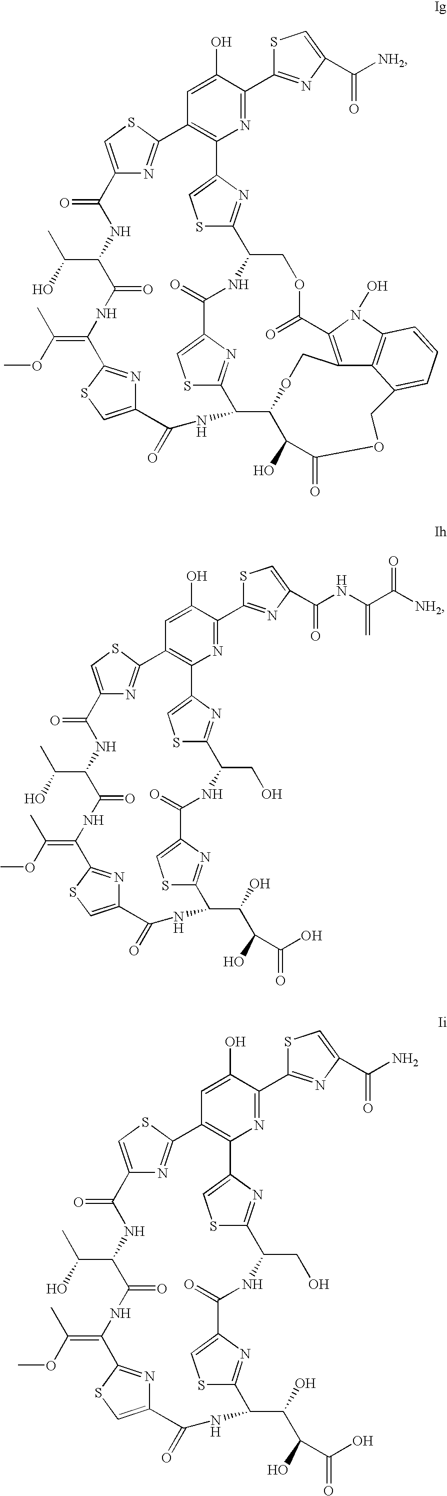
or pharmaceutically acceptable salts, esters, enantiomers, diastereomers or a mixture thereof.

3. A process for the preparation of the compound of structural formula I of claim 1, which comprises the cultivation of a Nocardia spp. with ATCC accession number 202099 or a natural or artificial mutant thereof in a nutrient medium and recovering the compound of structural formula I from the fermentation broth.

4. The process of claim 3 wherein the fermentation is conducted at a temperature of about 10° C. to about 40° C.

5. The process of claim 4, wherein the fermentation is conducted at a temperature of about 31-32° C.

6. A pharmaceutical composition comprising a pharmaceutically acceptable carrier and an effective amount of a compound of structural formula I of claim 1.

7. Use of a compound of formula I of claim 1 for the manufacture of a medicament for treating a bacterial infection in a host in need of such treatment.
